(19) United States
(12) Patent Application Publication
(10) Pub. No.: US 2013/0059297 A1
(43) Pub. Date: Mar. 7, 2013

(54) METHOD FOR ISOLATING RENAL STEM/PROGENITOR CELLS, RENAL STEM/PROGENITOR CELLS AND THERAPEUTIC AGENT FOR RENAL DISEASE

(71) Applicant: Bio Link, Incorporated, Minato-Ku (JP)

(72) Inventors: Takeshi Sugaya, Hyogo (JP); Naoshi Shinozaki, Tokyo (JP)

(73) Assignee: Bio Link, Incorporated, Minato-Ku (JP)

(21) Appl. No.: 13/659,635

(22) Filed: Oct. 24, 2012

Related U.S. Application Data
(63) Continuation of application No. 12/671,474, filed on Jul. 20, 2010, now abandoned.

(51) Int. Cl. C12N 5/071; C12Q 1/68

(52) U.S. Cl. 435/6.1; 435/325

(57) ABSTRACT

It is intended to provide a method for noninvasively isolating human renal stem/progenitor cells, an isolated renal stem/progenitor cells, a therapeutic agent for renal disease, mouse mesenchymal cells which can be used for isolating human renal stem/progenitor cells and a culture supernatant of the same. Renal stem/progenitor cells are isolated by primarily culturing cells contained in the urine of a patient having renal disease in a medium containing mouse mesenchymal cells identified by the deposition number of FERM ABP-10865 or a culture supernatant of the same, staining the obtained primarily cultured cells with Hoechst 33342 and separating a weak-positive or negative fraction.

### RELATED APPLICATIONS

This application is a continuation of U.S. application Ser. No. 12/671,474, filed Jul. 20, 2010, which is the U.S. National Phase filing under 35 U.S.C. §371 of PCT/JP2007/065375, filed Aug. 6, 2007.

### TECHNICAL FIELD

The present invention is related to a method for isolating human renal stem/progenitor cells, isolated renal stem/progenitor cells, a therapeutic agent for renal disease, mouse mesenchymal cells which can be used for isolating human renal stem/progenitor cells and a culture supernatant of the same.

### BACKGROUND ART

In recent years, the number of patients suffering from chronic renal disease is increasing. Chronic renal disease is a pathological condition which progresses from renal damage caused by a renal disease or a disease in an organ other than the kidneys and which causes the failure of normal renal functions such as the function of removing uremic toxins contained in the blood, the endocrine function and the adjustment function.

When chronic renal disease progresses, the loss of functions normally performed by the kidneys affects other internal organs and causes uremia. More specifically, central nervous system damage, peripheral neuropathy, disorders of the heart and circulatory system, disorders of the digestive system, disorders of the vision and eyes, disorders of the blood and clotting functions, immune disorders, disorders of the endocrine system, skin disorders, bone and joint disorders, electrolyte disorders and acid-base balance disorders may result.

Currently, the only radical treatment for chronic renal disease is kidney transplantation. However not all patients suffering from chronic renal disease can undergo transplantation treatment due to the lack of donors. As a result, patients suffering from chronic renal disease must undergo artificial dialysis in order to prolong their survival. The increase in dialysis patients causes an increase in medical costs and has become a large social problem. Although the technical advances in current artificial dialysis have reduced the load on a patient in comparison to previous techniques, there is a clear difference between the quality of life (QOL) of such patients and patients undergoing radical treatment by kidney transplant.
[Non-Patent Literature 1] Poulsom R. et al., “Bone marrow cells contribute to both normal turnover of renal epithelial and regeneration after damage.”, J. Pathol., 195, pp. 229-235, 2001.
[Non-Patent Literature 2] Bussolati B. et al., “CD133+ cells from kidney represent a multipotent adult resident stem cell population that may contribute to the repair of renal injury.”, Am. J. Pathol., 166, pp. 545-555, 2005.

### DISCLOSURE OF THE INVENTION

### Problems to be Solved by the Invention

However in recent years, regenerative therapies using stem/progenitor cells to form tissue or organs in vitro or in vivo have attracted considerable attention as a means of treating lesions or defects. This research trend has indentified the existence of renal stem/progenitor cells in the kidneys or marrow. (Refer to Non-Patent Literature 1, 2)

Although proposals have been made for the isolation and acquisition of renal stem/progenitor cells and their application to regenerative therapies, the isolation of renal stem/progenitor cells from the kidneys or bone marrow places a large burden on the body, that is to say, there are problems associated with the invasive procedures.

The present invention is proposed in light of the conventional situation and has the object of providing a method for noninvasively isolating human renal stem/progenitor cells, isolated renal stem/progenitor cells, a therapeutic agent for renal disease, mouse mesenchymal cells which can be used for isolating human renal stem/progenitor cells and a culture supernatant of the same.

### Means for Solving the Problems

The present inventors diligently carried out research from various points of view to achieve the above object. As a result, the present inventors realized that isolation and acquisition of renal stem/progenitor cells can be enabled by using urinary exfoliated cells which exfoliate into the urine of renal disease patients as a result of comprised renal function. The present invention was completed using this insight.

That is to say, the method of isolating renal stem/progenitor cells according to the present invention is characterized by obtaining a primary culture of cells contained in the urine of a patient having renal disease in a medium containing mouse mesenchymal cells identified by the deposition number of FERM ABP-10865 or a culture supernatant of the same, staining the obtained primary culture cells with Hoechst 33342 and isolating a weak-positive or negative fraction.

The mouse mesenchymal cells identified by the deposition number of FERM ABP-10865 are supporting cells in the primary culture. When preparing the primary culture of the urinary exfoliated cells, the specific mouse mesenchymal cells or a supernatant obtained by culturing the cells in a serum-containing medium is added to the medium to thereby enable extremely highly efficient isolation of renal stem/progenitor cells when compared to adding another supporting cells or a supernatant thereof.

In this context, although blood creatinine, cystatin C or BUN (blood urea nitrogen) are known as indicators of renal disease, in recent years, it has been reported that among the fatty acid binding proteins (FABP) contained in the urine or blood, liver-type fatty acid binding protein (hereafter “L-FABP”) is specifically expressed in the proximal renal tubules of the kidneys and therefore is capable of use as an indicator of renal disease (refer to Japanese Patent No. 3259758). In other words, although the upper normal limit for a urine L-FABP value is 10 ng/ml, L-FABP can be used as an indicator for renal disease since patients having renal disease present high values of 50 ng/ml or more for example. In particular, the present inventors have demonstrated a positive correlation between the ratio of weak positive/negative fractions in Hoechst 33342 and a urinary L-FABP value. Thus the present invention preferably uses L-FABP as an indicator to select a patient having renal disease.

Patients having renal disease in the present invention include patients immediately after surgical transplantation of an adult kidney and newborns with premature kidney function. This indicator can be used efficiently to isolate renal stem/progenitor cells from the urine since patients immediately after undergoing kidney transplantation surgery or newborns with premature kidney function display a high urinary L-FABP value (for example at least 100 ng/ml).

The renal stem/progenitor cells according to the present invention are characterized in being isolated using the above isolation method and the therapeutic agent for renal disease according to the present invention is characterized by including renal stem/progenitor cells isolated using the above isolation method.

Furthermore the present invention provides mouse mesenchymal cells identified by the deposition number of FERM ABP-10865 and a culture supernatant obtained by culturing the mouse mesenchymal cells in a serum-containing medium for the purpose of isolating renal stem/progenitor cells from the urine of a patient having renal disease.

### Effects of the Invention

The present invention provides isolation of renal stem/progenitor cells in a non-invasive manner from a patient having renal disease, the isolated renal stem/progenitor cells and a therapeutic agent for renal disease including the renal stem/progenitor cells. The present invention further provides mouse mesenchymal cells used for efficient isolation of the renal stem/progenitor cells and a supernatant of the same.

### BRIEF DESCRIPTION OF THE DRAWINGS

FIGS. 1A-1E show the results of microscopic observation over time of a culture of urinary exfoliated cells isolated from a patient having renal disease;

FIG. 2 shows cell domes formed by culturing urinary exfoliated cells;

FIGS. 3A and 3B show weak positive or negative fractions when the urinary exfoliated cells are stained using Hoechst 33342;

FIGS. 4A-4F show the results of microscopic observation over time of a culture of isolated SP cells which are weakly positive or negative to Hoechst 33342; and

FIG. 5 shows the relationship between the isolation ratio of SP cells and the urinary L-FABP value.

FIG. 6 shows the adhering of the isolated SP cells to the kidneys.

### PREFERRED MODE FOR CARRYING OUT THE INVENTION

The embodiments applying the present invention will be described in further detail hereafter making reference to the specific experimental results.

### Culturing of Urinary Exfoliated Cells

Firstly, urinary exfoliated cells are isolated from the urine of a patient having renal disease and cultured.

More specifically, 10 ml of urine from a patient immediately after kidney transplantation surgery was subjected to centrifugation for 5 minutes at 1100 rpm and 4° C. and a supernatant was discarded. The cells were suspended in 10 ml of DMEM (Dulbecco's Modified Eagle's Medium) (Sigma) containing 10% FBS (Fetal Bovine Serum) (MBL). The suspension was again subjected to centrifugation, the supernatant discarded and the cells isolated. The operation of cell purification by suspension, centrifugation, and discard of the supernatant was repeated four times. Cells isolated in the above manner were suspended in 10 ml of a medium being a 1:1 mixture of a medium in which DMEM/F12 (Sigma) with 10% FBS contains in final concentration 5 mg/ml insulin, 5 mg/ml transferrin, 5 ng/ml sodium selenite, 2.5 mg/ml nicotinamide, 10⁻⁸ M dexamethasone (all Sigma), and a culture supernatant prepared by culturing mouse mesenchymal cells identified by the deposition number of FERM ABP-10865 for approximately 24 hours in DMEM containing 10% FBS. Then the suspension was cultured on a gelatin-coated cell culture plate (BD FALCON). The mouse mesenchymal cells were deposited with the Independent Administrative Juridical Person: Industrial Technology Comprehensive Research Laboratories, Deposition Center of Microorganisms for Patent Application (Chuo 6^{th}, Tsukuba Center, Higashi 1-Chome 1-Banchi 1, Tsukuba-City, Ibaraki-Ken) on Jan. 20, 2006 and transferred to an international depositary on Jul. 10, 2007.

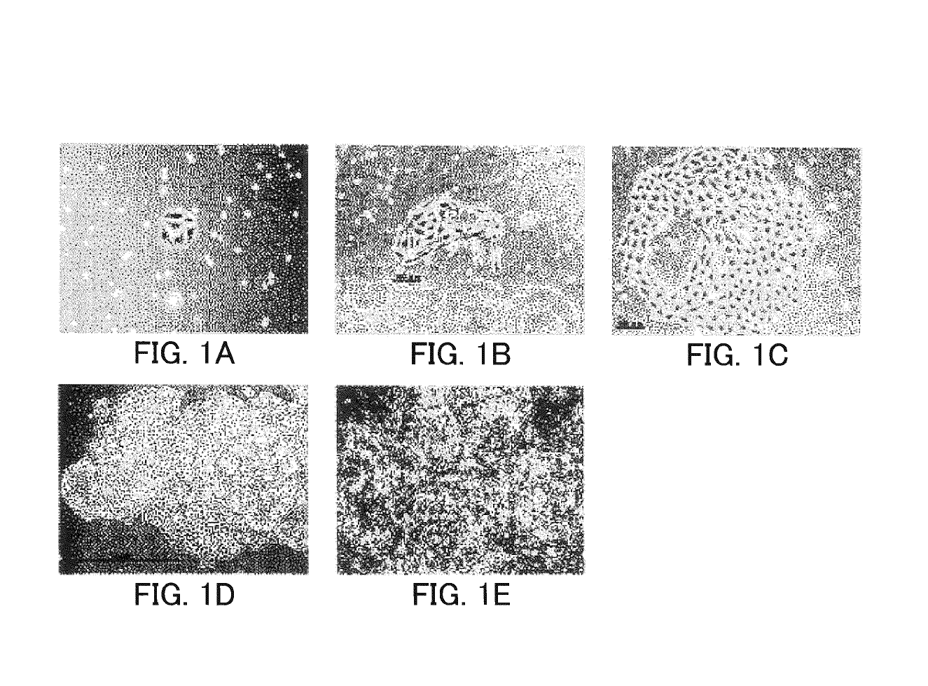

The results of microscopic observation of the cells over time are shown in FIGS. 1A-1E. FIGS. 1A-1E show the cells over time respectively at 3 days, 4 days, 6 days, 8 days and 15 days. The cells adhered to the culture plate within 24 hours after commencing culturing and cell division occurred on a 12-24 hour cycle thereafter to thereby result in a propagation of approximately 5×10⁶ cells in the two weeks after commencing culturing. These results enabled confirmation that the cells having a propagation capacity could be obtained using this culturing method.

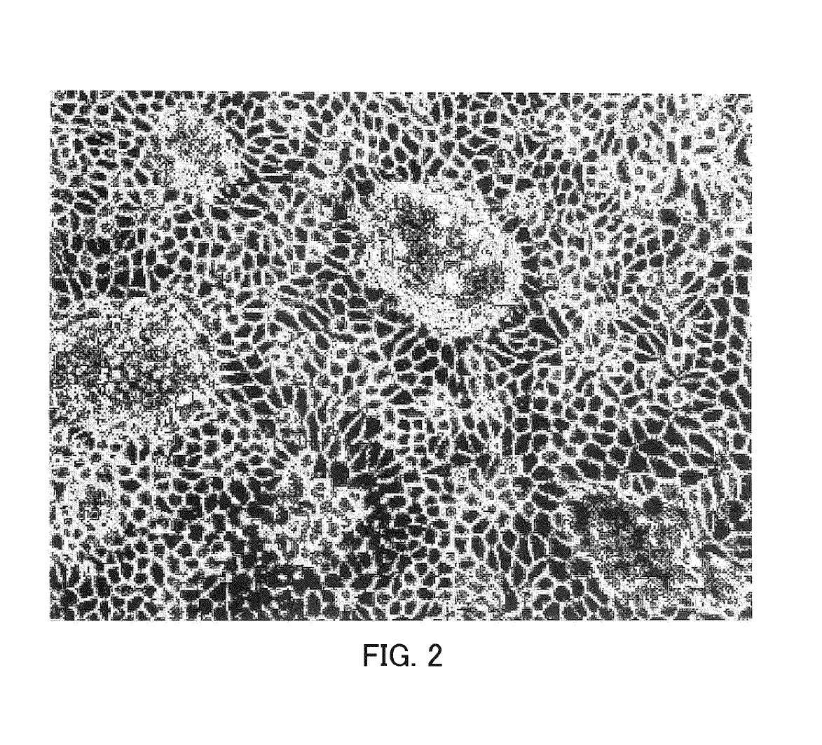

Colonies formed from nephric tubule epithelial-like cells displaying a tessellated morphology were observed in the cell groups with the above propagation capacity. As shown in FIG. 2, the formation of cell domes suggesting a transepithelial transport function was observed.

These results showed that acquisition and culturing of nephric tubule epithelial-like cells was enabled by culturing urinary exfoliated cells isolated from the urine of a patient immediately after undergoing kidney transplantation surgery.

### Genetic Expression Analysis of Urinary Exfoliated Cells

Next, genetic expression of the isolated urinary exfoliated cells was confirmed.

More specifically, a suspension was prepared by adding 1 ml of ISOGEN (Nippon Gene) to 1.5×10⁶ cells obtained by continuing the primary culture as described above for 5 weeks, then adding 2 ml of chloroform and mixing well. The resulting mixture was subjected to centrifugation for 10 min at 12,000 rpm and 4° C. After adding 0.5 ml of isopropanol to the supernatant and mixing well, centrifugation was applied for 20 minutes at 12,000 rpm and 4° C. Then the supernatant was discarded and the precipitate was washed in 1 ml of 75% ethanol and air-dried. The total RNA obtained in the above manner was sampled and genetic expression analysis was performed using a gene chip (Human Genome U133 Plus 2.0 Array Gene Chip, Affymetrix). Genes identified in this manner are shown in the Table below for both positive and negative results.

TABLE 1

| | | Proximal | Loop of Henle | | Distal | | Urinary |
|---|---|---|---|---|---|---|---|
| | | renal | Decending | Acending | renal | Collecting | exfoliated |
| | Glomerulus | tubule | limb | limb | tubule | duct | cells |
| Aquaporin 1 | | + | | | | | + |
| Aquaporin 2 | | | | | | + | − |
| K+/Cl— cotransporter, KCC3a | | + | | | | | + |
| Na+/HCO3— cotransporter, SLC4A4 | | + | + | | | | + |
| Na+/HCO3— cotransporter, SLC4A7 | | | | + | + | | + |
| Na+/HCO3— cotransporter, SLC4A1 | | | | | | + | − |
| Alkaline phosphatase, IAP | | + | | | | | + |
| Aminopeptidase A | | + | | | | | + |
| Calbindin D, 28K | | | | | + | | + |
| Nephrin | + | | | | | | − |
| E-cadherin | | + | | | + | + | + |
| N-cadherin | | + | | | | | + |
| P-cadherin | + | | | | | | − |
| K-cadherin | | + | + | + | | | + |

| | | |
|---|---|---|
| | Gene expression stage/area in kidney development | |
| Eya1 | metanephric mesenchyme | − |
| Lim1 | metanephric mesenchyme | − |
| Hoxa11 | metanephric mesenchyme → pretubular aggregate | + |
| WT1 | metanephric mesenchyme → immature glomerulus | − |
| Pax2 | metanephric mesenchyme → immature tubule | + |
| Pax8 | immature tubule | + |
| Foxd1 | stroma | + |
| Foxc1 | ureter bud | + |
| Emx2 | ureter bud | + |

As shown by Table 1, expression in the urinary exfoliated cells of genes specific to the proximal renal tubules was confirmed for genes including aquaporin 1, Na⁺/Cl⁻ cotransporter (KCC3a) and N-cadherin. Genetic expression was also confirmed for K-cadherin, and the Na⁺/CO₃⁻ cotransporter (SLC4A4) expressed in loop of Henle and the proximal renal tubules, the Na⁺/CO₃⁻ cotransporter (SLC4A7) expressed in the distal renal tubules and loop of Henle and calbindin D28K which is specifically expressed in the distal renal tubules. On the other hand, expression of genes specific for the collecting duct including the Na⁺/CO₃⁻ cotransporter (SLC4A1) or aquaporin 2 or genes specific for the glomerulus such as nephrin or P-cadherin was not confirmed. Genetic expression was confirmed for genes including Pax, Pax8 and the like which are expressed in the nascent fetal kidneys.

These results suggest the primary culture of cells from the urinary exfoliated cells contains undifferentiated cells such as renal stem/progenitor cells which are present in the nascent kidneys, or cells which have differentiated into nephric tubule epithelial-like cells of the renal nephron via the sequence of proximal renal tubules to loop of Henle to distal renal tubules.

Isolation of Renal Stem/Progenitor Cells from Urinary Exfoliated Cells

Hoechst 33342 which is a DNA binding stain is known to emit fluorescence in a range of at least 400-600 nm when excited by ultraviolet laser light. A heterogenous cell group containing a mixture of a plurality of cells types, for example bone marrow cells, was stained with this staining agent and then flow cytometry was used to develop the cells in two dimensions using blue fluorescence at approximately 450 nm and red fluorescence at approximately 675 nm. As a result, a cell group was indentified which displayed weak positive or negative results to Hoechst 33342 in a specific pattern in dark sections of fluorescence to a greater extent than the G0/G1 phase cells seen in normal cell cycle analysis. Since this cell group displays a development pattern of projecting from the main group (main population) containing the majority of residual cells, those groups are termed side population (SP) cells.

Bone marrow cells are known to have a concentration of hematopoietic stem cells in these SP cells (Refer to “Goodell M. A. et at., J. Exp. Med., vol 183, p. 1797, 1996”). In addition to the marrow, SP cells are found in tissues including the brain or skeletal muscle, the heart, liver or kidneys (Refer to Murayama A. et al., J. Neurosci. Res., vol. 69, p. 837, 2002; Gussoni E. et al., Nature, vol. 401, p. 390, 1999; Hierlihy A. M. et al., FEBS Lett., Vol. 530, p. 239, 2002; Asakura A. et al., Exp. Hematol., vol. 30, p. 1339, 2002; Iwatani H. et al., Kidney Int., vol. 65, p. 1604, 2004). These cells can also function as tissue stem cells.

It is considered that SP cells were not stained to a large degree by Hoechst 33342 since Hoechst 33342 was exported out of the cell by a pump-shaped molecule represented by proteins coded by MDR (multi drug resistance genes) which are a type of ABC transporter. Since the stem/progenitor cells display active MDR expression, the exportation function with respect to Hoechst 33342 suggests common properties in the stem/progenitor cells (Jikken Igaku, Vol. 19, No. 15 (Extra Edition), p. 68-73, 2001). The SP cellular fraction was completely eliminated by the addition of verapamil or reserpine which are function inhibiting agents of MDR molecular function (Proceedings of the 117^{th} Symposium of the Japanese Association of Medical Sciences, p. 66-74).

In this context, it was considered possible that SP cells were also present in the same manner in urinary exfoliated cells and functioned as renal stem/progenitor cells and therefore it was attempted to isolate SP cells from urinary sediment cells.

More specifically, urinary exfoliated cells after three weeks continuous primary culture in accordance with the above description were removed from the culture plate using an trypsin-EDTA solution (Invitrogen) and two suspensions with a concentration of 1×10⁶ cells/ml were prepared by suspending the cells in DMEM containing 2% FBS. Hoechst 33342 (Molecular probe) was added to each sample at a concentration of 5 mg/ml and a negative control was prepared by adding reserpine (Sigma) at a concentration of 1 mg/ml to only one of the samples. Thereafter, the cells were shake cultured for 1 hour at 37° C., subjected to centrifugation and the supernatant was discarded. The cells after discard of supernatant were suspended in HBSS (Hank's Balanced Salt Solution) containing 2% FBS, 10 mM HEPES (Invitrogen), 10² U/ml penicillin-streptomycin (Invitrogen) and having an addition of PI (propidium iodide) at a concentration of 1 mg/ml to screen dead cells. Then the SP cells were analyzed and isolated using a fluorescence-activated cell sorter (BD FACSAria, Beckton Dickinson).

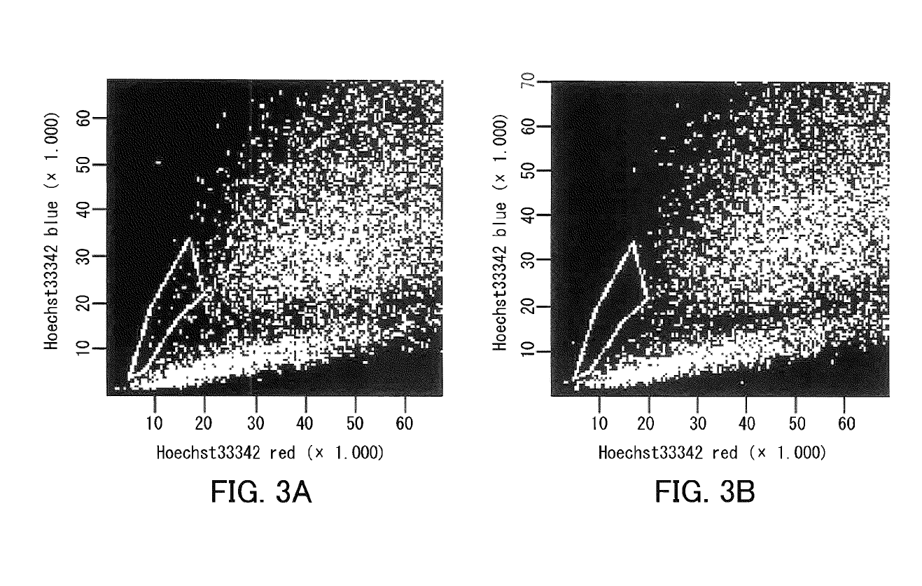

The analysis results for the SP cells are shown in FIGS. 3A and 3B. FIG. 3A shows a sample to which reserpine has not been added, FIG. 3B shows a negative control sample to which reserpine has been added. In the figures, the area encircled with the solid line depicts the Hoechst 33342 weak positive or negative fractions. These fractions are completely absent from the negative control containing reserpine and therefore demonstrate that such fractions are SP cell fractions. On average, 0.33% (n=6) SP cells were present in the urinary exfoliated cells.

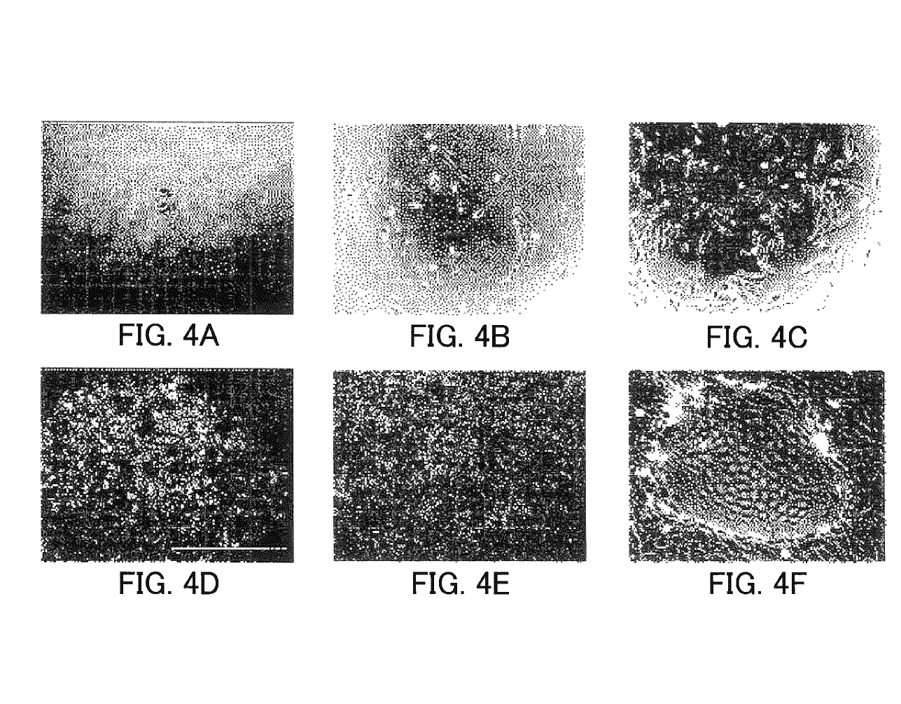

After isolation and acquisition of the SP cells, the cells were cultured using the same method as during primary culturing. The results of microscopic observation of the cells over time are shown in FIGS. 4A-4F. FIGS. 4A-4F show the cells after 12 hours, 3 days, 10 days, 15 days, 19 days and 22 days. When culturing, epithelial cell colonies having high propagation capacity and displaying proximal renal tubule epithelial-like tessellated morphology were obtained. Upon further culturing, formation of cell dome morphology was confirmed after 22 days. When cells other than SP cells were cultured using the same methodology, the formation of cell domes was not confirmed.

These results demonstrate that renal stem/progenitor cells which are differentiable into renal tubule epithelial cells can be isolated and acquired by isolating SP cells which are Hoechst 33342 weakly positive or negative from urinary exfoliated cells fin a primary culture.

Increasing Efficiency of Isolation of Renal Stem/Progenitor Cells from Urinary Exfoliated Cells

Next to examine increasing the efficiency of isolation of renal stem/progenitor cells from urinary exfoliated cells, the relationship of culture efficiency of the primary culture and the urinary L-FABP value which has been reported in recent years to be an indicator of renal disease was examined.

More specifically, urinary exfoliated cells isolated from the fresh urine of 239 patients including newborns with premature kidney function were used in a primary culture as described above and then the culture efficiency was compared with the urinary L-FABP value. Successful primary culture was defined as confirmation of epithelial-like cell colony formation after one week of culturing and propagation for three weeks. When the culture efficiency was compared with the urinary L-FABP value, the following results were obtained: Group 1: L-FABP<100 ng/ml, 15%; Group 2: 100<L-FABP<1000 ng/ml, 22%; Group 3: 1000<L-FABP<10000 ng/ml, 35%; Group 4: L-FABP>10000 ng/ml, 100%.

The primary culture cells were positive for alkaline phosphatase staining and the formation of cell domes was confirmed thereby demonstrating a transepithelial transport function. After using RT-PCR to confirm genetic expression by extracting mRNA from the primary culture cells, the expression of a proximal renal tubule specific amino transporter rBAT (related to bO,+system amino acid transporter) was confirmed.

These results demonstrate that use of urinary L-FABP as an indicator enables primary culture of urinary exfoliated cells which are differentiable into renal tubule epithelial cells from urine having a high L-FABP value.

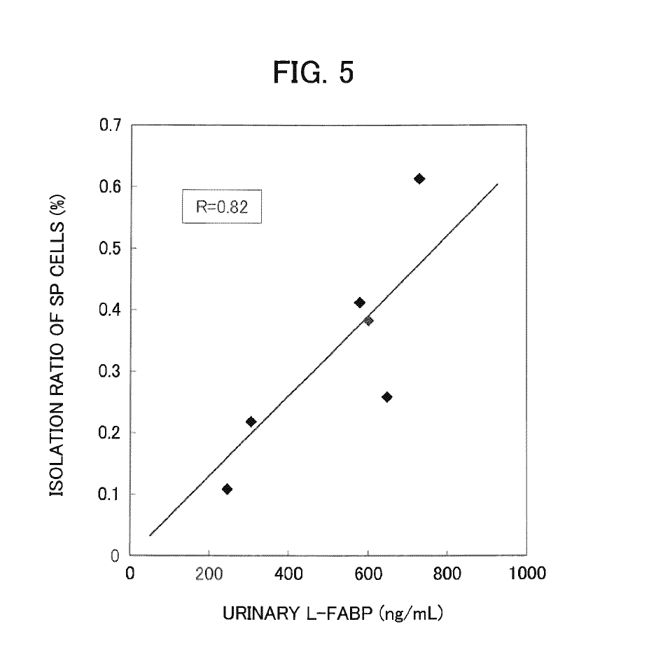

The relationship between a urinary L-FABP value and the differentiation efficiency for SP cells was also considered. These results clearly demonstrate that the ratio of SP cells as shown in FIG. 5 shows an extremely high correlation (R=0.82) with the urinary L-FABP value during primary culture.

This fact suggests that renal stem/progenitor cells can be acquired by specifying the period during which the urinary L-FABP value in patients having renal disease is increasing and collecting urine during that period. Consequently a method is provided which makes a considerable contribution to the non-invasive and efficient provision of human renal cells.

### Adhering of Renal Stem/Progenitor Cells to Kidneys

Next confirmation of whether or not the isolated stem/progenitor cells take to the adult kidneys and have a functional morphology was confirmed by considering whether or not the adult kidneys were adversely affected and whether or not the cells adhered during the kidney regeneration step.

More specifically, the recipient of heterografted human renal stem/progenitor cells was an experimental mouse (25 g) with impaired immunity thereby avoiding a tendency to undergo a rejection reaction. A model for ischemic acute renal failure was prepared by applying avascularization to the renal arteries and veins of both kidneys and releasing the ischemia after 30 minutes. After culturing the isolated SP cells for a further five weeks, a trypsin-EDTA solution (Invitrogen) was used to create a single cell dispersion and then cell implantation was performed using a syringe via injection into the subrenal capsule immediately after releasing the ischemia. An autopsy was performed after three days, the kidneys were removed and frozen sections were prepared.

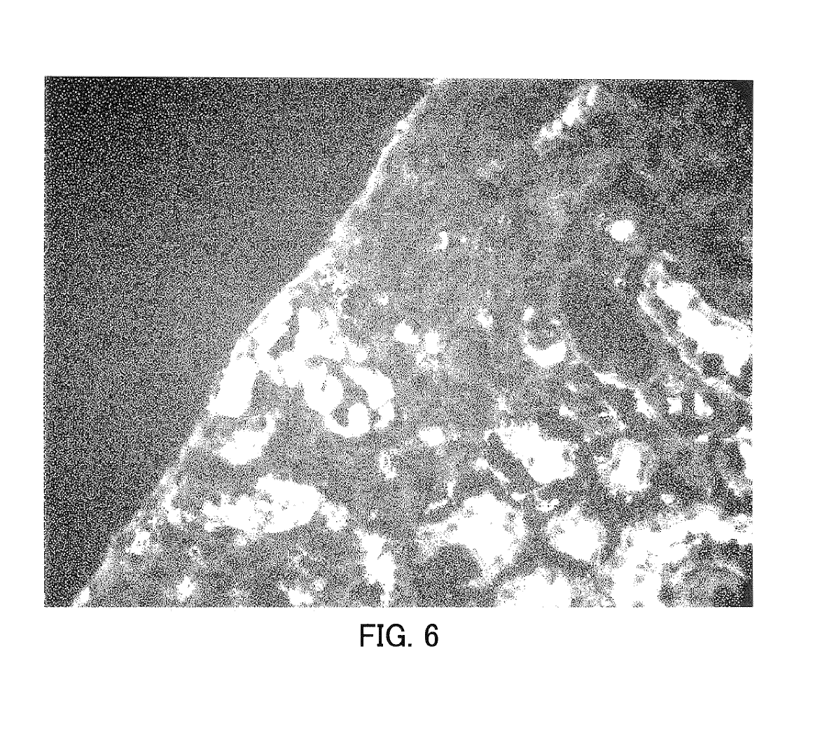

The results of double staining with an immunological stain using a human specific HLA antibody (green fluorescence indicator) and a nephric tubule specific aquaporin 1 antibody (red fluorescence indicator) are shown in FIG. 6. In the figure, the position indicated in yellow is the position of double positives at which the implanted cells form a nephric tubule-like lumen structure. Notwithstanding the fact that a heterograft was performed on the model for ischemic acute renal failure, no deterioration was observed with respect to the vital prognosis.

These results demonstrated that the isolated renal stem/progenitor cells take to the adult kidneys and have a morphology in which a protein is expressed which has a nephric tubule specific function. Thus it is considered that renal diseases can be treated by implantation of a therapeutic agent for renal disease including renal stem/progenitor cells into a patient having renal disease.

As described above making reference to the detailed experimental results, the method of the present embodiment enables non-invasive and highly efficient isolation of renal stem/progenitor cells from a patient having renal disease in addition to providing the isolated renal stem/progenitor cells. Furthermore a therapeutic agent for renal disease including the renal stem/progenitor cells can be provided in addition to the provision of specific mouse mesenchymal cells which can be used for isolating a human renal stem/progenitor cell and the supernatant of the same. In particular, since the method of the present embodiment enables isolation of renal stem/progenitor cells from a patient having renal disease, the isolated renal stem/progenitor cells can be implanted to that patient and thereby can be applied to the development of patient-specific treatments for renal disease. Furthermore a human nephric tubule cell culturing system obtained by culturing isolated renal stem/progenitor cells can be used for screening of pharmaceutical agents in vitro.

The present invention is not limited to the above embodiments and, of course, various modifications can be made without departing from the spirit of the invention.

1. A method for isolating renal stem/progenitor cells comprising:
identifying a sample of urine from a patient having renal disease exhibiting a urinary liver-type fatty acid binding protein (L-FABP) value of greater than 100 ng/ml;
preparing a primary culture of cells contained in said sample of urine from a patient having renal disease in a medium containing mouse mesenchymal cells identified by the deposition number of FERM ABP-10865 or a culture supernatant of the same; and
separating a weak-positive or negative fraction obtained by staining the cells in the primary culture with Hoechst 33342.

2. The method for isolating renal stem/progenitor cells according to claim 1, wherein the patient having renal disease is selected by using L-FABP as an indicator in urine of the patient.

3. The method for isolating a renal stem/progenitor cells according to claim 1, wherein the weak-positive or negative fraction is a fraction which is eliminated by addition of a function inhibiting agent for a MDR (multi drug resistance gene) molecule together with the Hoechst 33342.

4. A renal stem/progenitor cells isolated using the method of isolating renal stem/progenitor cells according to claim 1.

5. The renal stem/progenitor cells according to claim 4, wherein the renal stem/progenitor cells differentiates into a nephric tubule epithelial-like cell by culturing in a primary culture medium.

6. A therapeutic agent for renal disease comprising the renal stem/progenitor cells according to claim 4.

7. The method of claim 1, wherein said urinary L-FABP value is greater than 1000 ng/ml.

8. The method of claim 1, wherein said urinary L-FABP value is greater than 10,000 ng/ml.
